(19) United States
(12) Patent Application Publication
(10) Pub. No.: US 2004/0265341 A1
(43) Pub. Date: Dec. 30, 2004

(54) Prevention of uveitis

(75) Inventors: Thomas Lehner, London Bridge (GB); Miles R. Stanford, London (GB)

Correspondence Address: NIXON & VANDERHYE, PC, 1100 N GLEBE ROAD, 8TH FLOOR, ARLINGTON, VA, 22201-4714, US

(73) Assignee: Kings College London, London (GB)

(21) Appl. No.: 10/751,106

(22) Filed: Jan. 5, 2004

(30) Foreign Application Priority Data
Jun. 19, 2003 (GB) 0314360.9

(51) Int. Cl. A61K 39/02; C07K 14/28

(52) U.S. Cl. 424/236.1; 530/350

(57) ABSTRACT

A conjugate with recombinant cholera toxin B sub-unit (rCTB) of a peptide or polypeptide consisting of or containing a sequence corresponding to amino acid residues 336-351 of the human heat shock protein HSP 60, or the corresponding residues of the microbial 65 kD heat shock protein, or one which differs from either of these by up to and including 4 amino acid alterations (sub-situation and/or deletion and/or insertion) and having similar tolerising properties for Behcet's disease, or related types of uveitis, by oral, nasal, transmucosal or parenteral administration, or one which is extended from any one of the above-mentioned residues at the N-terminus or C-terminus or both with one or more non-wild-type amino acid sequences.

This invention relates to the treatment or prevention of uveitis, an inflammatory disease of the eye caused by an immune response to certain infective agents. Uveitis is a prominent feature of Behcet's disease (BD), a multi-system inflammatory disorder also characterized by oral and genital ulcers, cutaneous, vascular, joint and neurological manifestations. The present invention is applicable to all forms of uveitis, especially in BD, as well as the associated clinical manifestation and will be described below with particular reference to uveitis. BD is prevalent in countries bordering the Mediterranean and in the Middle and Far East especially Japan, China and Korea and is a significant cause of blindness in these countries.

The microbial 65 kD heat shock protein and the homologous human-HSP60 are found in a variety of microorganisms, such as Streptococcus sanguis, which have been implicated in the aetiology of Behcet's disease. A number of peptides present in these proteins have been identified by M. R. Stanford et al, Clin.Exp Immunol 1994 226-231, to which reference is hereby directed. Most of these peptides were shown in this reference to be uveitogenic when administered to rats, the two most effective being those with amino acids 336-351 and 136-150 of the aa sequence of HSP60. The 336-351 peptide had previously been identified by epitope mapping of mycobacterial HSP65, using overlapping peptides to stimulate T cell proliferation in BD (See Pervin K 1993 J.Immunol 151: 2273-2282 In this latter reference a homologous epitope was also identified within the sequence of human HSP60. The specificity of aa 336-351 as a major T cell epitope in BD was later confirmed by other workers. and used for diagnostic purposes (Hasan et al 1996, Lancet 347, 789-794) The significance of BD specific HSP peptide was greatly enhanced by the experimental evidence that uveitis can be induced in Lewis rats when the peptide was administered subcutaneously with an adjuvant (see Uchio E. et al 1998 Exp Eye Res 16 :174-180). Surprisingly, however, oral or nasal administration of p336-351 induced uveitis instead of tolerance, (see Hu. W et al 1998 European J.Immunol 28 :2444-2455) in contrast to retinal S antigen or IRBP administered orally in rats. This may be accounted for by having identified a specific uveitogenic epitope within the sequence of HSP60 and HSP65, unlike the whole retinal S antigen used in the latter experiment.

We have directed research to the possibility of suppressing the immunogenicity of the above mentioned peptides by chemically linking them to recombinant cholera toxin B subunit (rCTB). Recombinant CTB is primarily known as an adjuvant and, as such, its use for our purposes might have been contra-indicated. Although when rCTB is linked to certain proteins, the resulting conjugate has been shown to be useful as an oral tolerising agent in rodents (see Sun J-B et al 1994 Proc Natl Acad Sci 01: 10795-10799 and Sun J-B 2000 Int.Immunol 12: 1449-1457). This effect has not previously been demonstrated for relatively small immunogenic peptides given by the systemic route.

We have found that the 336-351 peptide-rCTB conjugate administered orally to experimental animals decreased significantly (p<0.0001) the development of uveitis (11/66, 16.7%), as compared with oral administration of the peptide alone (48/73, 65.8%) (see Phipps P A et al 2003 Eur.J.Immunol 33: 224-232, the contents of which are hereby incorporated by reference). The clinical and histological criteria of prevention of uveitis were associated with a significant increase in TH2 and decrease in TH1 type immune responses. We have also confirmed the value of this finding in human clinical trials provided certain conditions of treatment are followed, as will be described hereinafter. By contrast, this important therapeutic effect is not shown with the corresponding conjugate obtained with the 136-150 peptide used under the same conditions.

The 336-351 peptide used to prepare a conjugate in accordance with the present invention has the following amino acid sequence:—QPHDLGKVGEVIVTKD

The corresponding peptide from HSP65 (residue numbers 311-326) has the sequence:—DLSLLGKARKVVVTKD

This peptide may be used as an alternative to the HSP60 336-351 peptide.

For greater ease of conjugation we have added an N-terminal cysteine residue and a C-terminal acetate group to the above sequence to give:—CQPHDLGKVGEVIVTKD-acetate.

It will be appreciated however that minor amino acid differences from the above are permissible for conjugation according to the present invention. For example the peptide may differ from either of the above by up to and including 4 amino acid alterations (substitution and/or deletion and/or insertion or one which is extended from any one of the above-mentioned residues at the N-terminus or C-terminus or both with a non-wild-type amino acid sequence.

The conjugate will be administered as a pharmaceutical composition in a pharmaceutically acceptable carrier, preferably formulated for oral or nasal administration. The dose of conjugate may be in the range of from 0.1 to 20 mg per single dose administered across any mucosal membrane (e.g. mouth or nose) or orally, or by subcutaneous or intradermal injection.

The invention also comprises a method of treatment or prevention of Behcet's disease or related types of uveitis in patients which comprises administering an effective amount of the conjugate or composition defined above. Administration is preferably commenced after the patient has been free of disease activity for at least 2 and preferably up to 3-6 months before tolerisation is commenced. This is an important condition for successful use of the conjugates and is essential to avoid any recurrence of uveitis in BD and other forms of uveitis.

Conventional treatment of BD is by immunosuppressive agents, starting with a high dose of prednisolone and decreasing this to the minimal effective dose, with or without azathioprine or other drugs. We adopted the therapeutic approach first introduced for evaluation of tolerance with retinal S antigen in uveitis in humans, by oral administration of the antigen and progressive withdrawal of the immunosuppressive drugs. Our objectives were firstly to find out if the immunosuppressive drugs can be gradually withdrawn, whilst the patient receives the tolerising regimen of p336-351-CTB. The second aim was to find out if any of the patients who passed through the first phase without relapse of uveitis could be maintained disease-free, after the tolerising regime was also withdrawn and the patient was without any treatment. Indeed, oral administration of 0.5 mg or 5 mg of the peptide-rCTB conjugate 3×weekly for 12-16 weeks to 8 unselected patients with BD allowed us to withdraw their immunosuppressive drugs in 5 of the 8 patients without a relapse of uveitis. However, 2 of the 3 patients in whom uveitis relapsed, showed disease activity before the tolerisation regime was initiated. If we then consider only the 6 patients who were clinically controlled by their immunosuppressive drugs prior to the peptide-CTB treatment, 5 of the 6 patients had no relapse of uveitis after withdrawal of all immunosuppressive drugs. In the second phase of the trial, when the tolerising regime was also withdrawn, 2 of the 5 patients have been free of any manifestations of BD for over a year, whilst the other 3 developed uveitis within 1 to 10 months after cessation of tolerisation. Prevention of relapse of uveitis was associated with prevention of some of the extra-ocular features of BD, such as cutaneous, oral and genital manifestations. Associated with prevention of uveitis was a decrease in peptide-specific CD4⁺ T cell proliferation, a decrease in the proportion of CD4⁺ T cells, expression of TH1-type cells and IFN-γ production, in contrast to increased immune activity in those patients in whom uveitis relapsed.

The methodology to be used in accordance with the invention is to be consistent with the relevant parts of the clinical trial described below,

Patients and Method

Selection criteria, treatment regime and monitoring of patients Patients for this study were recruited from the Uveitis Clinic at St Thomas' Hospital. All patients satisfied the International Study Group criteria for the diagnosis of Behcet's Disease and all had a history of panuveitis requiring systemic immunosuppression for its control. At enrolment, all patients were male, had quiet eyes for at least 3 months and a history of relapse on attempting to reduce the dose of systemic treatment in the previous year. Some patients (see below) showed signs of systemic activity (recurrent oral ulcers and folliculitis) at entry to the study. We specifically excluded patients who had had active eye disease within the previous 3 months, pregnant women or those not on adequate contraception and children. All patients signed informed consent for the study which received local Ethical Committee approval by the St Thomas' Research Ethics Committee. Four patients were started on 0.5 mg and 4 patients on 5 mg of the peptide-CTB given orally 3×per week, dissolved in 100 ml bicarbonate solution whilst maintaining their previous immunosuppressive drug treatment (Table 1). At week 3, the latter was reduced in a stepwise manner, starting with prednisolone and then any other systemic drug (azathioprine, cyclosporine, etc), with the aim of tailing off all treatment by week 12. During this time the tolerising regime of peptide-CTB was maintained. The patients were then observed for a further 12 weeks at 4 weekly intervals. Subsequent follow up was based on clinical need or every 2 months. At each visit patients had a full ophthalmological examination with dilation (MRS) as well as a complete physical examination (TL). Details of ocular and systemic inflammation were recorded and changes in these signs were scored as previously described (Lancet 1982 ii 787-92). Patients also had optical coherence tomography images (4 mm horizontal scan length through the fovea), to determine the presence of retinal thickening or macular oedema. For the purpose of this study a relapse was defined as an increase in intra-ocular inflammation that required either re-starting or an increase in systemic immunosuppressive therapy. Accordingly, patients who experienced mild anterior uveitis controllable on steroid drops alone remained in the study.

Ten patients were recruited to the study, age range 29-36 years (except for 1 patient who was 51 years old; Table 1). However, 2 of these patients dropped out during the first and second month of study either due to travel abroad or non-compliance in the drug regime and are not considered further.

All patients were HLA typed, as described before (1997 Tissue Antigens 50:100-111).

1. Preparation of GMP Peptide 336-351 Covalently Linked to Human HSP60

The BD peptide corresponding to residues 336-351 of the human HSP60 and with an N-terminal Cys residue was synthesized in its acetate form under GMP conditions (H-Cys-Gln-Pro-His-Asp-Leu-Gly-Lys-Val-Gly-Glu-Val-Il2-Val-Thr-Lys-AspOH acetate) by Bachem (Bachem AG, Bubendorf, Switzerland). This peptide was coupled to a highly purified, recombinant CTB (rCTB) produced under GMP by SBL Vaccine (Stockholm, Sweden), commonly used as a component of the internationally registered oral cholera vaccine Dukoral. We used the bifunctional cross-linking reagent N-succinimidyl 3-(2-pyridyl)-dithio) propionate (SPDP) (Pierce Rockford, Ill., USA), as recommended by the manufacturer to ensure the coupling of 4-5 peptide residues 336-351 per rCTB pentamer. An aliquot of 500 μg CTB in 0.05 M sodium phosphate (2.5 mg/ml) at pH 8.6 was incubated with 42 μl SPDP for 30 minutes at room temperature and the pH was then adjusted to 7.5. Peptide 336-351 (200 mg) was dissolved in 40 ml PBS, mixed with CTB-SSPY and incubated over night at room temperature on a rocking platform. After conjugation, the material was chromatographed on a Sephadex 25 column (Amersham Biosciences, Uppsala, Sweden) and eluted with PBS. The absorbance was recoded at 280 nm, and the protein fractions were collected and pooled. After checking that this material gave a single homogeneous peak it was eluted as a slightly larger protein than unconjugated SPDP-treated rCTB on FPLC. The rCTB-peptide conjugate was sterile-filtered, aliquoted, frozen and then stored. Selected aliquots of the final material were characterized for their sterility, composition, retained binding GM1 ganglioside and also tested for its safety and preventive capacity in the rat uveitis model as reported before being used in the present phase I/II clinical trial.

2. Haematological and Biochemical Tests

Routine blood indices and chemistry were carried out at every visit of the patients.

3. Immunological Investigations

a) Reagents

For analytical purposes HSP65 was prepared from M. bovis as described elsewhere (Mehlert A and Young D B. 1989. Biochemical and antigenic characterisation of the Mycobacterium tuberculosis 71 kD antigen, a member of the 70 kD heat-shock protein family. Mol. Microbiol. 3:125-130.).

The peptide 336-351 and control peptide 136-151 were synthesized in the Hansen's Disease Laboratory (Centre for Disease Control, Atlanta, Ga.) as described before (Pervin K et al 1993 J.Immunol 151: 2273-2282).

b) T Cell Proliferative Responses

PBMC were separated from defibrinated blood by density gradient centrifugation. The cells were cultured (10⁵ cells/well) with the optimal concentration of p336-351, p136-151, HSP65, Concanavalin A (Sigma) and without any antigen in quadruplicates for 5 days in 96-well round-bottomed plates, in a humidified atmosphere (5% carbon dioxide at 37° C.). The culture medium contained penicillin 100 μg/mL, streptomycin 100 U/mL, 2 mmol/L L-glutamine and 10% autologous serum. The cultures were pulsed with tritiated thymidine (18.5 mBq per well) in the final 6 h of incubation. They were harvested and then assessed for ³H thymidine incorporation by liquid scintillation counting. The results were assessed in stimulation indices, defined as the ratio of counts per minute of antigen stimulated to unstimulated cultures.

c) Studies of Cell Surface Markers by Flow Cytometry

Phenotypic analysis of cell surface molecules was carried out by diluting whole blood (1:1) with phosphate-buffered saline, 1% bovine serum albumin and 0.1% sodium azide (PBA); 50 μl of diluted blood was then mixed with 50 μl of the appropriate fluorochrome-conjugated antibodies. After 30 minutes, the red cells were lysed by adding two ml of FACS lysing solution (Becton Dickinson, Oxford, UK). Five minutes later the cells were washed with PBA and analysed on a Coulter XL counter (Coulter, Oxford, UK). Cells for analysis were gated by cell size (forward and size scatter), so as to analyse the lymphocyte population, except for CD40 expression the cells were gated on monocytes. The only exception was the analysis of CCR7 for which an unconjugated antibody (Cat No. 550937, BD Biosciences, Oxford) was used in combination with FITC-conjugated goat anti-mouse IgM antibody (Southern Biotech Associates, Alabama, USA).

d) Cytometric Bead Analysis of IFN-γ and TNF-α

The human Th1/Th2 Cytokine Cytometric Bead Array method (BD Biosciences Pharmingen, San Diego, Calif.) was used to assay TNF-α, IFN-γ IL-2, IL-4, IL-5, IL-10 and concentrations in culture supernatants from PBMC stimulated with either p336-351 or HSP65 for 3 days (as above). The assays were performed essentially according to the manufacturer's instructions and analysed in a BD Calibur flow cytometer with data analysis carried out by the BD CBA Analysis software.

Results

Clinical findings The investigation was completed on 8 patients, all of whom were maintained prior to being enlisted to this clinical trial, on a minimal effective dose of immunosuppressive drugs. Any attempt over the previous year to decrease prednisolone by even 2.5 mg had precipitated a relapse in uveitis. About 3 weeks after starting oral tolerisation with p336-351-CTB, all immunosuppressive drugs were gradually withdrawn over a period of 6-10 weeks. There was no detectable difference in the response of patients given 0.5 mg as compared with 5 mg of the peptide-CTB conjugate. Three of the 8 patients showed a relapse of uveitis within 1-2 months, whereas in the remaining 5 patients we were able to withdraw all immunosuppressive drugs, whilst receiving p336-351-CTB (Table 1). None of the patients reported any adverse effect, and their blood pressure, haematological indices and blood chemistry showed no significant changes.

Further analyses of these patients revealed that 2 of the 3 patients who experienced a relapse of uveitis showed muco-cutaneous disease activity at the start of the trial, with recurrent aphthous stomatitis and extensive pustular skin lesions. Thus, they were not controlled adequately by the immunosuppressive treatment. However, considering only those patients who showed no muco-cutaneous or ocular disease activity at the start of trial, 5 of the 6 patients were controlled by the peptide-CTB treatment, after the immunosuppressive drugs had been withdrawn (Table 1). Interestingly, the third patient who relapsed was of Middle Eastern origin, was well maintained on the lowest dose of prednisolone alone (5 mg daily), was much older (51 years) than the other patients (29-39 years and had long-standing BD (>13 years). The 5 patients who responded to the tolerising regime, showed no evidence of relapsing uveitis or muco-cutaneous lesions, except that in 1 of the 5 patients mild oral ulceration recurred and 1 of 3 patients the arthralgia showed no improvement. The results of the first phase of the trial showed that in 5 of the 6 patients in whom the disease activity was controlled by immunosuppressive drugs prior to initiating the trial, these drugs could be completely withdrawn whilst the patients were maintained on the oral tolerisation regime. In the second phase of the trial we were anxious to find out if the disease would remain in remission after the p336-351-CTB tolerising regime was withdrawn and the patient left without any treatment (Table 1). Two of the 5 patients developed uveitis within one month of discontinuing the peptide-CTB regime and 2-3 months after total withdrawal of the immunosuppressive drugs. However, one of the 5 patients remained free of uveitis and other manifestations of BD for a period of 10 months and 2 patients for over a year after discontinuing the tolerisation regime and withdrawal of all immunosuppressive drugs. It is noteworthy that 1 of the 2 patients free of BD manifestations for over 1 year was of Middle Eastern origin. Altogether the 5 patients with BD who were maintained disease-free during the tolerising regime and without any immunosuppressive treatment were in good health and have lost their cushingoid appearance.

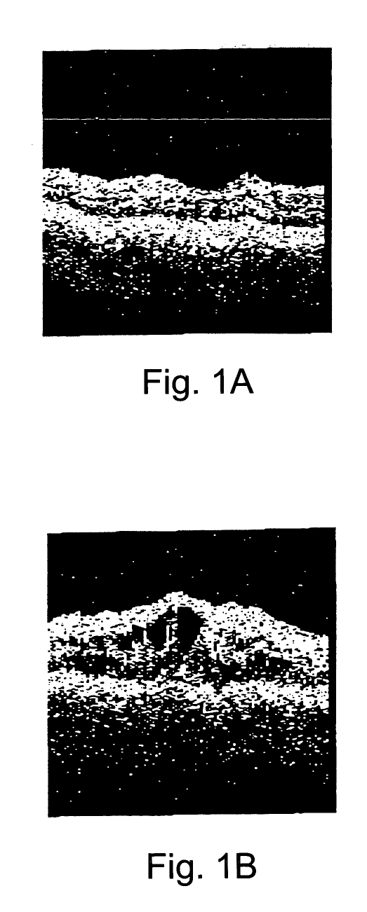

Optical coherence tomography images showed normal anatomical features of the fovea during remission, in contrast to gross macular oedema and retinal thickening in a relapse of uveitis (FIG. 1). Retinal thickness correlated directly with visual acuity; in remission 20/20, in exacerbation 20/200.

Immunological Changes

a) HLA

All but 1 patient expressed the HLA B51.101 allotype.

b) T Cell Proliferation

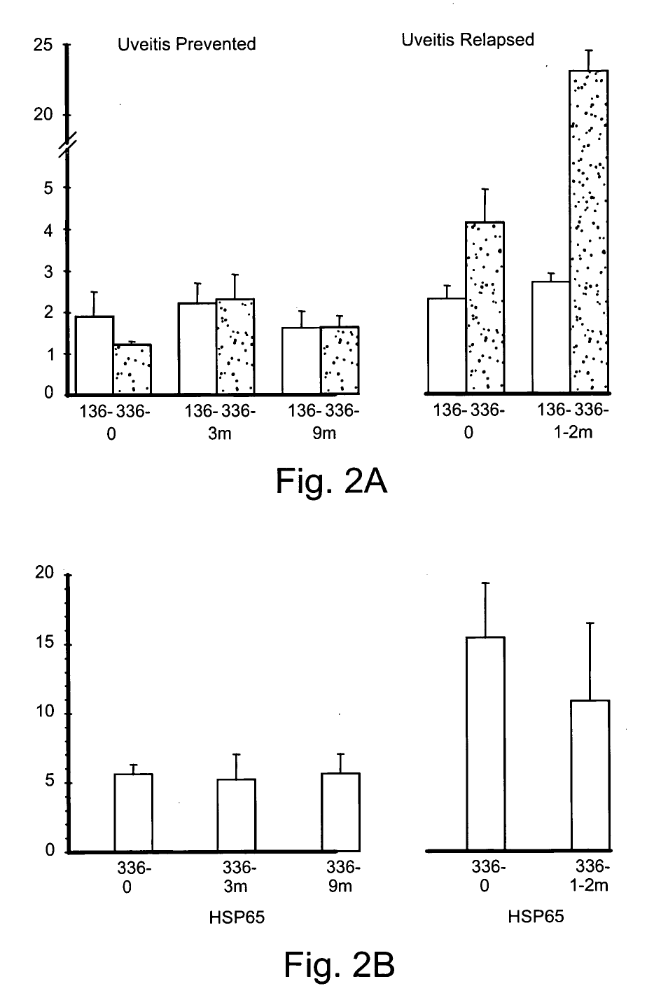

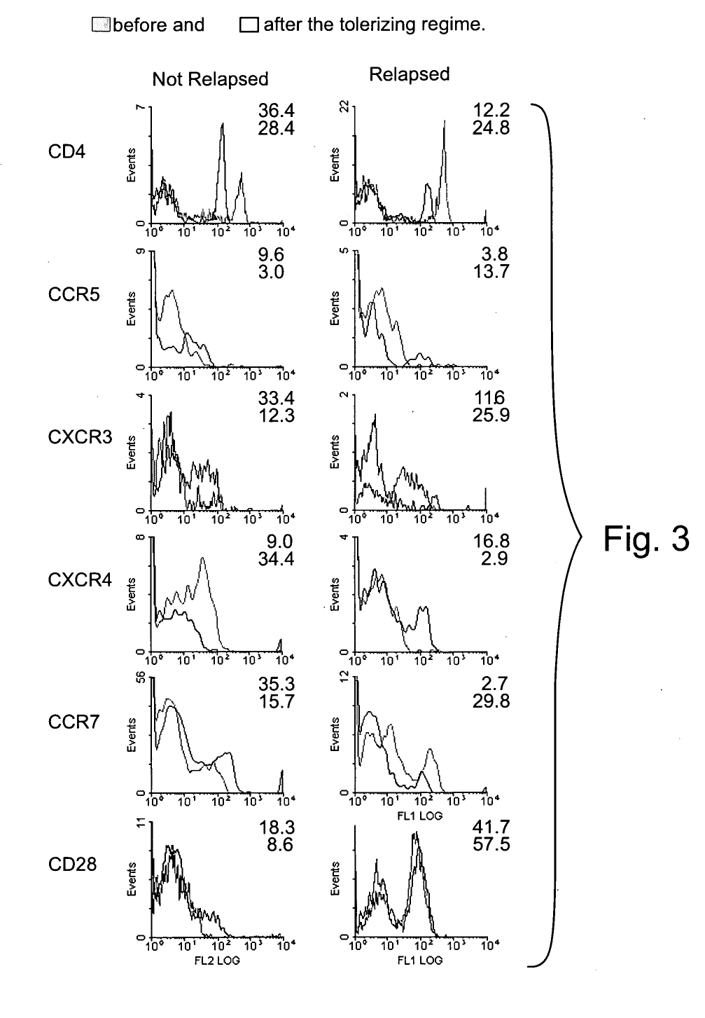

Stimulation with the BD specific peptide 336-351 showed SI<2 before the tolerising regime started in the 5 patients in whom the development of uveitis was prevented (mean±sem 1.25±0.1). In contrast, all 3 patients with relapsing uveitis showed a SI>2 (4.1±0.8) before the start of the tolerising regime (FIG. 2). This was consistent with disease activity in 2 of the 3 patients in the latter group, as shown in Table 1 (No. 7 and 8), but in none of the group of 5 patients in whom uveitis was prevented by the tolerising treatment. Sequential monitoring the T cell proliferative response in the latter group stimulated by p336-351 showed only a slight increase in the proliferative response to p336-351 (SI of 2.3±0.6) 3 months after the tolerising regime was started and all pre-existing immunosuppressive drugs were tailed off (FIG. 3A). The control peptide 136-151 elicited a similar change, and by the end of 9 months after all treatment had ceased both peptides induced SI<2 (FIG. 3A). However, in the 3 patients with relapsing uveitis 1-2 months after the tolerising regime was initiated, when the doses of pre-existing immunosuppressive drugs were decreased, the p336-351 response rose from a mean of 4.1 (±0.8) to 23.2 (±14.8), as compared with the control peptide 136-151 of 2.3 (±0.3) to 2.7 (±0.2). The tolerising regime was discontinued and the immunosuppressive treatment restarted (2 of them with 30 mg prednisolone and either cyclosporine A or mycophenolate mofetil), and monitoring their T cell proliferative responses was discontinued.

HSP65 was significantly raised before the tolerising regime was started in all 8 patients, but higher mean SI were found in relapsing uveitis (15.4±3.9) than in the group in which uveitis was prevented (5.6±0.7), which is consistent with disease activity in the former group (FIG. 1). However, this response has not reached the 5% level of significance in this or in previous investigations of BD⁶. There was little or no change in response to HSP65 after oral tolerisation in either group of patients. Concancavalin A response was a positive control in all lymphoproliferative assays and was very high (SI>50).

c) Phenotypic Analysis

The objective was to monitor sequentially the proportion of cells which might show differences between patients in whom the tolerising regime elicited a remission in uveitis as compared with those in whom a relapse developed (Table 2). The flow cytometric changes are presented before and after the tolerisation regime in 2 representative patients, one of whom showed no relapse in uveitis (FIG. 3) and the other developed uveitis as soon as the dose of immunosuppressive drugs was decreased. The proportion of CD4⁺ cells decreased in ⅘ subjects with a remission from a mean of 42.8 (±3.4)% to 26.4 (±2.2)% over the 3 months of p336-351-CTB administration and decreased further to 22.0 (±2.2)%, 6 to 9 months after treatment had ceased (Table 2A). In contrast, patients who developed relapsing uveitis showed 28.6 (±8.6)% CD4⁺ cells before which increased to 46.1 (±10.4)% during administration of the tolerising regime. Similar inverse relationships between the 2 cohorts was found in the expression of CCR5 and CXCR3 which are markers of TH1⁺ cells, CCR7 a memory T cell homing to lymph nodes and CXCR4 which with CCR7 is upregulated in dendritic cell maturation. CD28 and CD40 which are essential costimulatory molecules binding B7 and CD40L respectively showed similar changes (Table 2A). A potential difference was also found with TCR γδ⁺, CCR6⁺ and CD86⁺ cells but the inverse relationship was much less marked between the 2 groups of patients (Table 2B). However, CD8, CD45RA, CD45RO and CCR3 showed little or no difference between the 2 groups of patients.

Cytometric Bead Analysis of IFN-γ and TNF-α

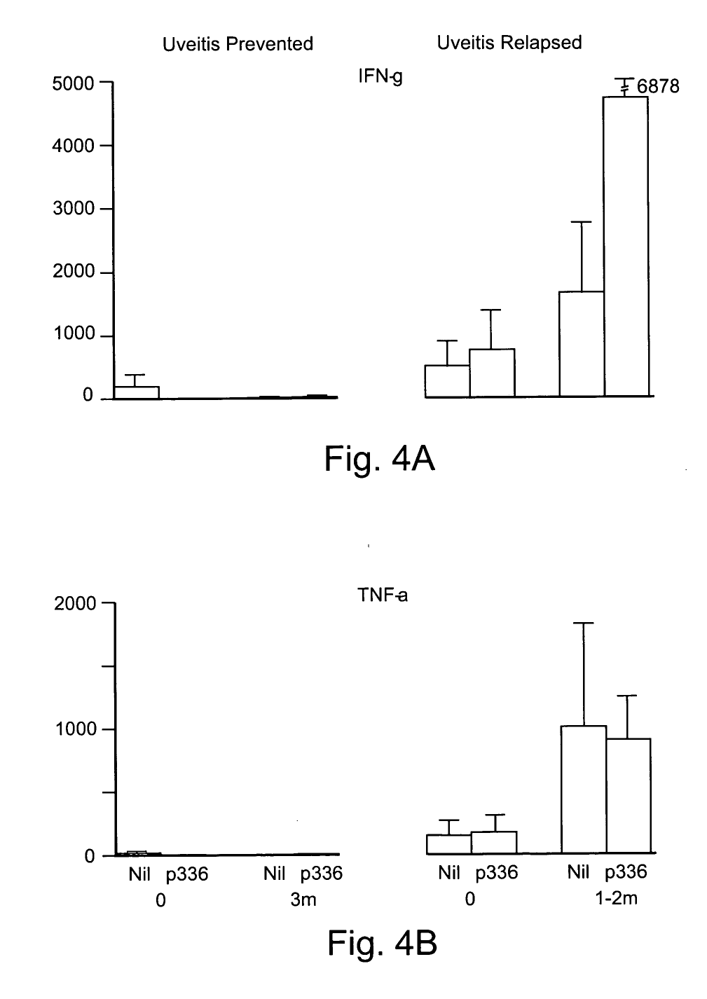

Specific stimulation with p336-351 involved negligible production of IFN-γ in those patients in whom the tolerising regime prevented uveitis, both before and 3 months after the immunosuppressive drugs were withdrawn (FIG. 4A). In contrast, increased concentration of IFN-γ was found in the group of patients in whom uveitis relapsed, even before tolerisation started; without stimulation (504±411 pg/ml) and with p336-351 stimulation (758±636 pg/ml). This increased greatly to 1665±1111 pg/ml without stimulation and to 4723 (±2639) with p336-351 stimulation 1-2 months after tolerisation was initiated and the immunosuppressive drugs were in the process of being decreased (FIG. 4A). Similarly, whereas TNF-α concentrations were negligible in the protected patients, increased levels of TNF-α were found mostly 1-2 months after tolerisation and the patients developed uveitis (FIG. 4B). These results suggest that an increase in both IFN-γ and TNF-α concentrations are associated with development of uveitis. Stimulation with HSP induced production of IFNγ and TNF-α, but this was found both in the protected and relapsing groups of uveitis, though the levels of IFN-γ and TNF-α were somewhat lower in the relapsing group of uveitis (data not presented).

Discussion

A specific HSP epitope (p336-351) was first identified in patients with BD, followed by experimental induction of uveitis in rats by systemic (Stanford M. R. et al, 1994 Clin. Exp. Immunol. 97: 226-231) and then oral administration (Hu W et al 1998 European J. Immunol 28: 2444-2455) of the peptide. Experimental uveitis could be prevented by oral administration of the p336-351-rCTB conjugate (Phipps P. A et al, ref above). In 5 out of 8 unselected patients with BD who were treated by immunosuppressive drugs, administration of the BD-specific conjugate prevented relapse of uveitis during and after the drugs had been withdrawn. The finding that 2 of the 3 patients who suffered a relapse of uveitis using this tolerising regimen had not been controlled with immunosuppressive drugs and showed evidence of muco-cutaneous disease and some ocular activity is consistent with the experimental evidence in animals. This demonstrated that whereas significant reduction in the development of uveitis was found on oral tolerisation with the conjugate before or on the day of oral immunisation with the peptide alone (p<0.0001), uveitis was not prevented if the conjugate was applied after the start of immunization with the peptide. Thus, in animal experiments BD-specific conjugate prevented the development of uveitis only if it was started before the disease was induced, and in humans a relapse was prevented only when adequate suppression of disease activity by immunosuppressive drugs had been elicited prior to administration of the tolerising regime.

It should be noted that in all patients attempts to decrease the dose of prednisolone by even 2.5 mg during the previous year had resulted in a relapse of uveitis. It is noteworthy that the third patient in whom uveitis relapsed after a decrease from 5 to 2.5 mg prednisolone on alternate days, whilst on the tolerising treatment for 4 weeks, was the oldest patient (51 years), as compared with 29 to 36 years of the other patients. The duration of BD was also longer than in most of the other patients. This suggests that the peptide-rCTB immunotherapy might be most effective in patients with a shorter duration of disease. Our secondary objective was to find out if patients who passed through the first phase without relapse might maintain their remission after the tolerising regimen was withdrawn and the patients were left without any further treatment. Indeed, 2 of the 5 patients have been maintained for over a year and 1 patient for 10 months after the immunosuppressive and then tolerisation treatment was discontinued. The remaining 2 patients relapsed within 1 month of cessation of the tolerisation regimen.

Of considerable interest was the effect of immunotherapy on the extra-ocular manifestations of BD. We found that only 1 of the 5 patients developed recurrent oral ulcers, and arthralgia of the knees improved in 2 of the 3 patients, though this was a minor feature in the disease. Folliculitis or erythema nodosum in 2 patients or genital ulcers in 1 patient did not recur. As BD manifestations and possibly severity might differ in the Middle East, it should be noted that 1 of the 2 patients who was free of any clinical features of BD for over 1 year after all immunosuppressive and tolerisation treatment were discontinued was a Turkish man. We should emphasize that there were no clinical adverse effects and the routine haematological indices and biochemical analyses showed no significant changes.

It should be noted that ⅞ patients HLA-typed expressed the HLA-B51.101 haplotype. This is consistent with the report that English BD patients with ocular manifestations express HLA-B51, as do patients in Japan and Turkey. The immunological investigations showed good correlation with the clinical changes in patients who maintained a remission and in those who suffered a relapse in BD when treated with the peptide-CTB conjugate. The T cell proliferative responses to p336-351 in the group of patients in whom uveitis was prevented were absent (SI<2) before and, with some fluctuation, remained low or absent during and after oral tolerisation. This was in contrast to the group of patients with relapsing uveitis who started with a significantly raised T cell proliferative response to p336-351 (4.1±0.8) which rose to 23.2 (±14.8) with the relapse of disease manifestations. The specificity of the response to p336-351 in BD has been established before and again demonstrated here with little or no stimulation with the unrelated HSP60 peptide 136-151. In addition to the striking difference in BD specific T cell responses to p336-351 there was also a remarkable difference in a number of immune parameters between the 2 groups of patients. Thus, the mechanism of tolerance appears to result in a decrease in TH1 type cells expressing CCR5⁺ and CXCR3⁺ and memory T cells expressing CCR7, decreased or absent IFN-γ and TNF-α production, and the 336-351 specific T cell response, and a decrease in the proportion of cells with the costimulatory CD28 and CD40 phenotypic expression.

This novel oral tolerisation regime, used for the first time in humans, established first of all safety of administration of the peptide-CTB conjugate. We have further defined selection criteria of BD patients for the tolerisation strategy in that the disease should be of short duration (<13 yrs), the patients under 45 yrs of age and the patients should be adequately controlled with immunosuppressive drugs, so as to be free of disease activity for at least 3 months prior to starting the tolerisation regimen. We have identified a number of immunological parameters that differentiate BD patients in remission and exacerbation. We have identified 3 types of responders: those patients who do not develop relapsing uveitis whilst on the tolerising regime, those that relapse within 10 months and those that do not relapse for over a year after discontinuing tolerisation.

Legend to Tables and Illustrations

Table 1. Clinical features and treatment of the patients taking part in the phase I clinical trial.

Table 2. Phenotypic analysis of PBMC in the two groups of patients with BD in whom uveitis was either prevented or relapsed during and after the change from immunosuppressive drugs to p336-351-CTB tolerising regime.

FIG. 1. Optical coherence images of the fovea (4 mm horizontal scan) of a) patient in remission after all immunosuppressive treatment has been withdrawn and b) patient in relapse. The retina is markedly thickened and shows black cystic spaces consistent with cytoid macular oedema.

FIG. 2. T cell proliferative responses stimulated with p336-351 or 136-151 (A) and HSP65 (B) before (0) and after tolerance was complated (3 months) and at the end of observation (9+ months) in 5 patients in whom uveitis was prevented and 3 patients with relapsing uveitis.

FIG. 3. Phenotypic analysis of 6 cell surface markers by flow cytometry in 2 representative patients undergoing tolerising regime, in one of whom uveitis was prevented in contrast to the other in whom uveitis relapsed.

FIG. 4. The effect of stimulating PBMC with p336-351 on the production of IFN-γ and TNF-α, evaluated by cytometric bead analysis.

1. A conjugate with recombinant cholera toxin B sub-unit (rCTB) of a peptide or polypeptide consisting of or containing a sequence corresponding to amino acid residues 336-351 of the human heat shock protein HSP 60, or the corresponding residues of the microbial 65 kD heat shock protein, or one which differs from either of these by up to and including 4 amino acid alterations (sub-situation and/or deletion and/or insertion) and having similar tolerising properties for Behcet's disease, or related types of uveitis, by oral, nasal, transmucosal or parenteral administration, or one which is extended from any one of the above-mentioned residues at the N-terminus or C-terminus or both with one or more non-wild-type amino acid sequences.

2. A conjugate according to claim 1, having an added N-terminal cysteine residue and a C-terminal acetate group.

3. A conjugate according to claim 1 or 2, prepared with the use of N-succinimydyl 3-(2-pyridyl)-dithio) propionate as cross linking agent.

4. A conjugate according to claim 3, containing 4 or 5 peptide residues per mol of rCTB pentamer.

5. A pharmaceutical composition comprising the conjugate of claim 1, in a pharmaceutically acceptable carrier.

6. A composition according to claim 5, which is formulated for oral or nasal or transmucosal administration, or for subcutaneous or intradermal administration.

7. A method of treatment or prevention of Behcet's disease or related types of uveitis in patients which comprises administration of an effective amount of the conjugate or composition of claim 1.

8. A method according to claim 7, in which a dosage of from 0.1 to 20 mg of the conjugate is administered per single dose.

9. A method according to claim 8 in which the dose of the conjugate is in the range of from 0.1 to 5.0 mg.

10. A method according to claim 7, in which administration is commenced after the patient has been free of disease activity for at least 2 and preferably 3-6 months before tolerisation is commenced.

11. A method according to claim 10, in which the patient has had adequate suppression of disease for up to 6 months by immunosuppressive or other treatment before tolerisation is commenced.


SEQUENCE LISTING (3 sequences)

SEQ ID NO: 1 - protein, 16 aa, Homo sapiens
QPHDLGKVGEVIVTKD

SEQ ID NO: 2 - protein, 16 aa, Mycobacterium sp.
DLSLLGKARKVVVTKD

SEQ ID NO: 3 - protein, 17 aa, Artificial Sequence
  Description of Artificial Sequence Synthetic peptide
CQPHDLGKVGEVIVTKD